(19) United States
(12) Patent Application Publication
(10) Pub. No.: US 2024/0371527 A1
(43) Pub. Date: Nov. 7, 2024

(54) METHOD FOR EXTRACTING COHORT, COHORT EXTRACTING APPARATUS AND COHORT EXTRACTING PROGRAM IMPLEMENTING THE METHOD

(71) Applicant: KaKao Healthcare Corp., Seongnam-si (KR)

(72) Inventors: Daehyeob RYU, Seoul (KR); Yuna LEE, Seoul (KR)

(21) Appl. No.: 18/566,741

(22) PCT Filed: May 11, 2022
(86) PCT No.: PCT/KR2022/006743

§ 371 Date: Dec. 4, 2023

(30) Foreign Application Priority Data
Jun. 7, 2021 (KR) 10-2021-0073385

(51) Int. Cl. G16H 50/70; G06F 17/40

(52) U.S. Cl. CPC G16H 50/70; G06F 17/40

(57) ABSTRACT

Disclosed is a method of operating a cohort extracting apparatus, the method including: obtaining cohort entry criteria and extracting events corresponding to the cohort entry criteria from a clinical data warehouse; generating an initial history table including an event identifier, a patient identifier, and a bit string indicating satisfaction of criteria of an initial step for each extracted event; obtaining criteria of a current step, identifying, among patients included in a history table of a just previous step, current step patients having an event corresponding to the criteria of the current step, and updating a bit string for each event of the current step patients included in the history table of the just previous step, and generating a history table of the current step by adding new events extracted in the current step; and sequentially generating a history table for each step, and then generating a cohort table by using a history table of a final step.

### TECHNICAL FIELD

The present disclosure relates to patient cohort extraction.

### BACKGROUND ART

Cohort extraction is very critical because researchers use cohorts extracted from Clinical Data Warehouses (CDWs) to conduct medical research. Thus, the researcher tries to determine whether a cohort that satisfies the various criteria is appropriate and to extract a cohort of the appropriate number of patients by varying the criteria.

However, a cohort extracting apparatus in the related art receives input of criteria, and outputs a patient group satisfying all criteria from the CDW, and the number of extracted patients varies depending on the criteria. As a result, the researcher has to repeat the cohort extraction operation on the vast CDW as criteria change, which takes a significant amount of time before the researcher obtains the satisfactory cohort. Additionally, as the number of criteria increases, the query volume increases, and it is necessary to re-extract patients with unchanged criteria, so that unnecessary operations are repeated.

### DISCLOSURE

### Technical Problem

The present disclosure attempts to provide a method of extracting a cohort in steps, and a cohort extracting apparatus and a cohort extracting program implementing the same.

Specifically, the present disclosure attempts to provide a method of extracting a cohort by generating a history table including events of each patient in each step, and updating a bit string indicating whether criteria are satisfied for each event in the history table.

### Technical Solution

An exemplary embodiment of the present disclosure provides a method of operating a cohort extracting apparatus, the method including: receiving input of cohort entry criteria and extract events corresponding to the cohort entry criteria from a clinical data warehouse; generating an initial history table including an event identifier, a patient identifier, and a bit string indicating satisfaction of criteria of an initial step for each extracted event; receiving input of criteria of a current step, identifying, among patients included in a history table of a just previous step, current step patients having an event corresponding to the criteria of the current step, and updating a bit string for each event of the current step patients included in the history table of the just previous step, and generating a history table of the current step by adding new events extracted in the current step; and sequentially generating a history table for each step, and then generating a cohort table by using a history table of a final step.

Each history table generated in each step may include events that satisfy criteria of a corresponding step, and may be written with an event identifier, a patient identifier, and a bit string indicating whether the criteria have been satisfied up to the corresponding step for each event. The bit string may be assigned a position of 1 or 0 to indicate whether the condition of each step is satisfied.

The generating of the history table of the current step may include checking the events of the current step patients in the history table of the just previous step, updating bit strings of the checked events with a value representing the satisfaction of the criteria of the current step, and recording the updated bit string in the history table of the current step.

The generating of the history table of the current step may include: when a new event is extracted from the current step, recording an identifier of the new event, a patient identifier, and a bit string indicating the satisfaction of the criteria of the current step in the history table of the current step. The bit string of the new event may be written with a value of 1 for digits specified in the current step, and a value of 0 for digits specified in other steps.

The generating of the history table of the current step may include, among the patients included in the history table of the just previous step, identifying a previous step patient who does not have an event corresponding to the criteria of the current step, and not recording events of the previous step patient in the history table of the current step.

The method may further include, when a request for the number of events or the number of patients extracted from a specific step is received, calculating said number of events or said number of patients by using a history table of said specific step.

The method may further include: receiving input of change criteria of a specific step; retrieving a just previous step history table generated in a just previous step of the specific step; and identifying, among patients included in the just previous step history table, specific step patients having events corresponding to the change criteria of the specific step, updating a bit string for each event of the specific step patients included in the just previous step history table, and regenerating a history table of the specific step by adding new events extracted in the specific step.

The method may further include sequentially regenerating a history table of a step after the specific step by using the regenerated history table of the specific step.

Another exemplary embodiment of the present disclosure provides a method of operating a cohort extracting apparatus, the method including: receiving input of criteria; based on clinical data of patients included in a first history table generated in a just previous step, identifying a current step patient satisfying the criteria among the patients included in the first history table; recording event identifiers, a patient identifier, and an updated bit string of all events of the current step patient included in the first history table in a second history table; when a new event corresponding to the criteria is extracted, recording an event identifier of the new event, a patient identifier, and a bit string representing an event extracted in the current step in a second history table; and storing the second history table as a history table of the current step.

For all events of the current step patient included in the first history table, a bit string in which a value of a digit specified to the current step in the bit string recorded in the first history table is updated to 1 may be recorded in the second history table.

For the new event, a bit string in which a value of a digit specified to the current step is 1 and a value of a digit specified in other steps is 0 may be recorded in the second history table.

Among the events included in the first history table, events of a previous step patient having no event corresponding to the criteria may not be recorded in the second history table.

Still exemplary embodiment of the present disclosure provides a computer program stored in a computer-readable storage medium and including instructions executed by at least one processor, the instructions being described to execute: receiving input of cohort entry criteria and extract events corresponding to the cohort entry criteria from a clinical data warehouse; generating an initial history table including an event identifier, a patient identifier, and a bit string indicating satisfaction of criteria of an initial step for each extracted event; receiving input of criteria of a current step, identifying, among patients included in a history table of a just previous step, current step patients having an event corresponding to the criteria of the current step, and updating a bit string for each event of the current step patients included in the history table of the just previous step, and generating a history table of the current step by adding new events extracted in the current step; and sequentially generating a history table for each step, and then generating a cohort table by using a history table of a final step.

Each history table generated in each step may include events that satisfy criteria of a corresponding step, and may be written with an event identifier, a patient identifier, and a bit string indicating whether the criteria have been satisfied up to the corresponding step for each event. The bit string may be assigned a position of 1 or 0 to indicate whether the condition of each step is satisfied.

The generating of the history table of the current step may include: checking the events of the current step patients in the history table of the just previous step, updating bit strings of the checked events with a value representing the satisfaction of the criteria of the current step, and recording the updated bit string in the history table of the current step; and when a new event is extracted from the current step, recording an identifier of the new event, a patient identifier, and a bit string indicating the satisfaction of the criteria of the current step in the history table of the current step.

### Advantageous Effects

According to the exemplary embodiments, the events of each patient extracted for each step and the bit string indicating whether each event satisfies the criteria for each step is managed as a history table, it is possible to quickly calculate the number of patients and the number of events at each step by using the plurality of history tables, thereby allowing researchers to quickly determine the adequacy of the cohort.

According to the exemplary embodiments, through the bit string indicating whether each event satisfies the criteria for each step, it is possible to quickly check the step at which the event was extracted and the step at which the event satisfies the criteria.

According to the exemplary embodiment, after completing the extraction of events up to the final step, when the criteria of a specific step need to be changed, it is possible to generate a new history table including events that satisfy change criteria by using the history table generated in the just previous step.

### DESCRIPTION OF THE DRAWINGS

FIGS. 1 and 2 are diagrams illustrating a cohort extracting method in the related art.

FIG. 3 is a diagram illustrating a cohort extracting apparatus.

FIGS. 4 to 6 are diagrams illustrating an example of a cohort extracting method.

FIG. 7 is a diagram illustrating a cohort re-extracting method using a history table.

FIG. 8 is a flowchart illustrating the cohort extracting method.

FIG. 9 is a hardware diagram of a computing apparatus according to an exemplary embodiment.

### MODE FOR INVENTION

Hereinafter, exemplary embodiments of the present invention will be described with reference to accompanying drawings so as to be easily understood by a person ordinary skilled in the art. The present disclosure can be variously implemented and is not limited to the following exemplary embodiments. Accordingly, the drawings and description are to be regarded as illustrative in nature and not restrictive. Like reference numerals designate like elements throughout the specification.

Throughout the specification, unless explicitly described to the contrary, the word “comprise”, and variations such as “comprises” or “comprising”, will be understood to imply the inclusion of stated elements but not the exclusion of any other elements. In addition, the terms “-er”, “-or”, and “module” described in the specification mean units for processing at least one function and operation, and can be implemented by hardware components or software components, and combinations thereof.

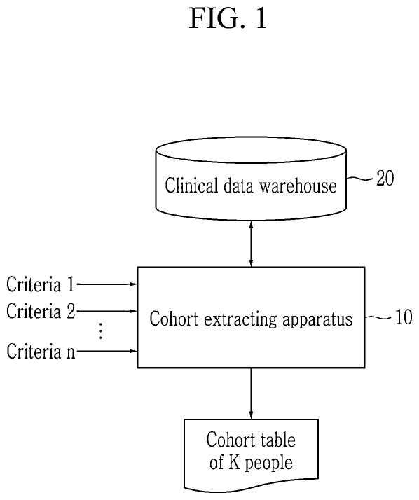

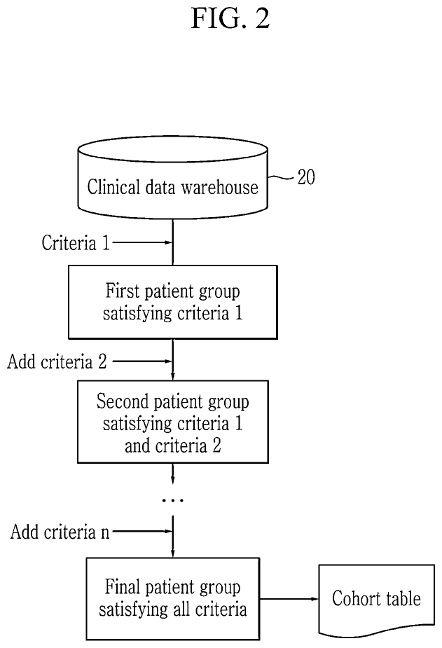

FIGS. 1 and 2 are diagrams illustrating a cohort extracting method in the related art.

Referring to FIG. 1, a cohort extracting apparatus 10 in the related art receives input of cohort criteria (criteria 1, criteria 2, . . . , criteria n) from a researcher and extracts K patients who satisfy all criteria from a Clinical Data Warehouse (CDW) 20 that stores various patient data. The cohort extracting apparatus 10 in the related art outputs a cohort table containing data from K patients.

When a researcher wants to change criteria 1 or delete criteria 1, the researcher may input changed criteria into the cohort extracting apparatus 10 in the related art and obtain a cohort of M patients who satisfy all criteria. However, when even any of the input criteria is changed, the cohort extracting apparatus 10 in the related art has to perform the cohort extraction operation again, so that the cohort extraction operation is repeated, and it is necessary to extract the patients with unchanged criteria again, which causes the repeat of unnecessary operations. Also, as the number of criteria increases, the query volume increases, which may significantly increase the extraction time.

Referring to FIG. 2, the cohort extracting apparatus 10 in the related art may receive input of the cohort criteria (criteria 1, criteria 2, . . . , criteria n) from the researcher in steps and extract K patients while gradually reducing the number of patients. In other words, the cohort extracting apparatus 10 in the related art may extract a group of K patients by extracting a first patient group satisfying criteria 1, extracting a second patient group satisfying criteria 2 from the first patient group, extracting a third patient group satisfying criteria 3 from the second patient group.

The patient group extracted from each step includes the patients who satisfy all the criteria up to the corresponding step, so the researcher may obtain patients who satisfy all the criteria set from the initial step to the current step. As such, the cohort extracting apparatus 10 in the related art is focused on extracting patients, and identifies only the patients who satisfy all of the criteria up to the current step (for example, diagnosed with hypertension, 50s, male, prescribed drug A, prescribed drug B). Thus, the researcher simply can know that the extracted patients satisfy all the criteria up to the current step (for example, diagnosed with hypertension, 50s, male, prescribed drug A, prescribed drug B), but has difficulty in knowing whether the patient was prescribed drug A and drug B together or separately, or whether drug A was prescribed for a diagnosis of hypertension or for another disease. When a researcher wants to get a cohort in which the patients were prescribed with both drug A and drug B together, it is necessary to analyze the patient data and re-select the patients.

On the other hand, when there is a single attribute desired to be searched, such as a keyword search, a search apparatus only needs to extract a desired target from one-dimensional data. However, even though the cohort extraction operation is extracting from clinical data of one patient, it needs to fetch data suitable to criteria from tables for each attribute, such as age, gender, primary diagnosis name, secondary diagnosis name, diagnosis date, administered drug name, and prescription date. As a result, in the cohort extraction operation, the search speed is exponentially slower depending on the size of the table, the nature of the attributes, and the search criteria, and when the operation needs to be repeated every time the criteria change, time and resources may be wasted.

The following describes a cohort extracting method including the improvement of the related art in more detail.

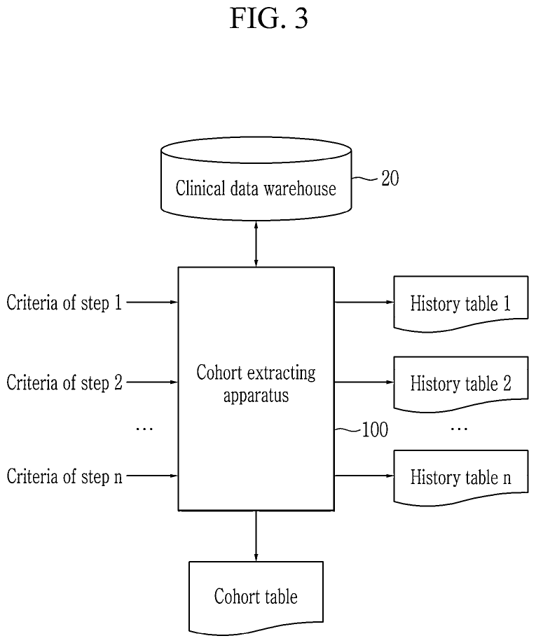

FIG. 3 is a diagram illustrating a cohort extracting apparatus.

Referring to FIG. 3, the cohort extracting apparatus 100 is a computing apparatus operated by at least one processor. The processor of the cohort extracting apparatus 100 performs the operations of the present disclosure by executing instructions included in a computer program. A computer program may include instructions that cause a processor to execute the operations of the present disclosure and may be stored on a non-transitory computer readable storage medium. Computer programs may be downloaded over a network or sold as a product, and installed on computing devices at various sites, such as laboratories and hospitals.

The cohort extracting apparatus 100 extracts cohorts from the CDW 20 that stores various patient data. Patient data extracted from the CDW 20 may be of various types, which are collectively referred to as clinical data for convenience. Further, the cohort extracting apparatus 100 may extract patient data from a variety of stores, which will be described as extracting patient data from the clinical data warehouse for convenience.

The cohort extracting apparatus 100 receives input of the criteria in steps, and extracts events that satisfy the criteria for each step, sorts the events by patient, and generates a history table including events of each patient. In this context, an event is information that can be checked in a CDW 20, and means information distinguishing an event, behavior, and the like that occurred to a patient at a specific point in time. For example, the event may be defined as a diagnosis event (for example, a history of a diagnosis of diabetes with an E10-E14 disease code), a drug prescription event (for example, a history of a prescription for aspirin), an examination event (for example, a history of having a low-density lipoprotein (LDL) cholesterol test), a hospitalization event (for example, a history of an emergency room visit), etc. Here, the criteria may include a cohort entry criteria (for example, people with at least one diagnosis of hypertension) and the detailed criteria you want to extract (for example, drug, and age). The detailed criteria may be defined as inclusion or exclusion of an item, and may be defined as a range.

The cohort extracting apparatus 100 initially generates history table 1 for the cohort entry criteria, and then separately generates history table 2, . . . , and history table n by using the input criteria in steps.

The history table contains a bit string that indicates whether the criteria have been satisfied up to the current step for each event, as 0 or 1. Each digit in the bit string is assigned a step, and a value of 1 for the corresponding bit indicates that the criteria of the corresponding step is satisfied, and a value of 0 for the corresponding bit indicates that the criteria of the corresponding step is not satisfied. For example, when the bit string is 10 bits, “0000000001” represents an event that satisfies the criteria in step 1, “0000000011” represents an event that satisfies the criteria in step 1 and step 2, and “0000000010” represents an event that satisfies the criteria in step 2.

The cohort extracting apparatus 100 identifies, among the patients included in the history table of the previous step, patients in the current step who have the event corresponding to the criteria in the current step. Then, the cohort extracting apparatus 100 generates a history table of the current step consisting of events that satisfy the criteria of the current step.

In this case, when the event of the patient in the current step exists in the history table of the previous step, the cohort extracting apparatus 100 updates the bit string of the corresponding event (for example, updates the bit string from “0000000001” to “0000000011”), and adds the event extracted from the current step as a new event to generate the history table of the current step. The new event may be written with a bit string in which the bit assigned to the current step is “1” (for example, “0000000010”).

The cohort extracting apparatus 100 identifies, from among the patients included in the history table of the previous step, a previous step patient who has no event corresponding to the criteria of the current step. Further, the cohort extracting apparatus 100 does not retrieve the events of the previous step patient from the history table of the previous step into the history table of the current step.

The history table is generated in each step, and is written in the unit of event, and depending on the patient, the plurality of events may be written, where the events of the patient who have at least one event corresponding to the criteria of the corresponding step are written. The schema for the history table may be defined in a variety of ways, for example, as shown in Table 1, where rows contain events, columns contain event information, and the history table is sorted for each patient. Event information may include a patient identifier (person_ID), visit identifier (visit_ID), event start date (start_date), event end date (end_date), event type (event_type), and detailed criteria type (criteria_type). Herein, the visit identifier (visit_ID), event start date (start_date), and event end date (end_date) may be used as event identifiers to distinguish between events

TABLE 1

| person_ID | visit_ID | start_date | end_date | event_type | criteria_type |
|---|---|---|---|---|---|
| A | 1 | 2021 Jan. 2 | 2021 Jan. 3 | 0000000011 | |
| A | 3 | 2021 Jan. 10 | 2021 Jan. 20 | 0000000010 | criteria1 |
| | | | | | (e.g., drug) |
| B | 5 | 2021 Feb. 1 | 2021 Feb. 7 | 0000000010 | criteria1 |
| | | | | | (e.g., drug) |
| B | 7 | 2021 Feb. 15 | 2021 Feb. 17 | 0000000011 | |

In Table 1, the patient identifier (person_ID) is an identifier that distinguishes patients who satisfy the criteria. The visit identifier (visit_ID) is an identifier that distinguishes the visit where the event occurred. The event start date (start_date) and the event end date (end_date) indicate the start date and the end date of the event. The event type (event_type) is the event's step information, which may be represented as a bit string of 0 or 1 indicating whether the criteria up to each current step have been satisfied, and may be updated based on the step. The detailed criteria type (criteria_type) is information that indicates the detailed criteria for which the event was extracted, and the detailed criteria for which the event has been initially extracted is written.

The cohort extracting apparatus 100 may calculate and output the number of patients and the number of events from the history table in each step. Thus, researchers may easily determine the adequacy of the extracted cohort by checking the number of patients and the number of events.

The cohort extracting apparatus 100 may quickly extract only events with a specific event type from the history table. For example, when the cohort extracting apparatus 100 extracts events with an event type of “********11” from the history table, the cohort extracting apparatus 100 may calculate the number of events that are caused by patients that satisfy criteria 2 among the events that satisfy criteria 1, and may calculate the number of patients having events that satisfy criteria 1 and criteria 2 based on the patient identifier of the event with “********11”. Thus, the cohort extracting apparatus 100 does not need to newly generate an SQL query to extract the event from the CDW to calculate the number of events or the number of patients, but may quickly calculate the number of events and the number of patients by performing a bitwise operation on the event type column of the history table.

The cohort extracting apparatus 100 may generate a cohort table from the history table in the final step or a specific step, and output the generated cohort table. The cohort table includes various clinical data for the patients included in the history table.

On the other hand, a researcher may want to change the criteria of a specific step after completing the event extraction up to the final step. In this case, the researcher inputs the specific step and the change criteria which he/she wishes to change with the cohort extracting apparatus 100. The cohort extracting apparatus 100 may then retrieve a history table generated in the just previous step of the specific step from among the stored history tables, and generate a new history table for the specific step that includes events that satisfy the change criteria by using the history table.

In the following, a method of generating, by the cohort extracting apparatus 100, the history table in steps will be described in detail.

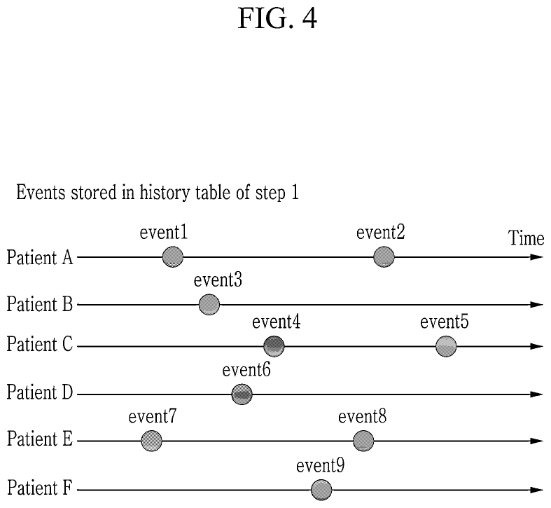

FIGS. 4 to 6 are diagrams illustrating an example of a cohort extracting method.

Referring to FIGS. 4 to 6, an example of a method of generating, by the cohort extracting apparatus 100, a history table in steps will be described.

Criteria of the initial step, step 1, are cohort entry criteria, which may be, for example, a person who has been diagnosed with hypertension at least once. It is assumed that the criteria in step 2 is drug. In step 2, events where a drug or a specific drug is prescribed are extracted. It is assumed that the criteria in step 3 is age. In step 3, patients in a specific age range are extracted.

First, referring to FIG. 4, the cohort extracting apparatus 100 receives input of the criteria of step 1 and extracts the hypertension diagnosis events corresponding to the criteria of step 1 from the clinical data warehouse (CDW). For example, it is assumed that nine events are extracted: event1, event2, . . . , event9, where event1 and event2 are hypertension diagnosis events for patient A, event3 is a hypertension diagnosis event for patient B, event4 and event5 are hypertension diagnosis events for patient C, event6 is a hypertension diagnosis event for patient D, event7 and event8 are hypertension diagnosis events for patient E, and event9 is a hypertension diagnosis event for patient F.

The cohort extracting apparatus 100 may store the events extracted according to the criteria of step 1 as history table 1, and record a patient identifier and an event identifier (visit identifier, event start date, event end date), along with a bit string indicating whether the criteria are satisfied up to the current step, in the event type. The cohort extracting apparatus 100 may generate a history table of step 1 as shown in Table 2. For convenience, the values for the event start date (start_date) and event end date (end_date) are omitted from the history table.

TABLE 2

| event | person_ID | visit_ID | event_type | criteria_type |
|---|---|---|---|---|
| 1 | A | 1 | 0000000001 | |
| 2 | A | 3 | 0000000001 | |
| 3 | B | 5 | 0000000001 | |
| 4 | C | 7 | 0000000001 | |
| 5 | C | 9 | 0000000001 | |
| 6 | D | 11 | 0000000001 | |
| 7 | E | 13 | 0000000001 | |
| 8 | E | 15 | 0000000001 | |
| 9 | F | 17 | 0000000001 | |

Referring to Table 2, since the events are extracted from step 1, “0000000001”, which has the last digit of 1 assigned to step 1 may be written in the event type. Since step 1 is the cohort entry criteria, the detailed criteria type, which represents the detailed criteria under which the event was extracted, has a null value (NULL).

when the cohort extracting apparatus 100 receives a request for the number of events extracted in step 1, the cohort extracting apparatus 100 may calculate the number of rows in the history table in step 1 that have the event type (event_type) of “0000000001” and output the number of events as 9.

When the cohort extracting apparatus 100 receives a request for the number of patients extracted in step 1, the cohort extracting apparatus 100 may calculate the number of patients distinguished by the patient identifier (person_ID) from the history table in step 1, and output the number of patients as 6.

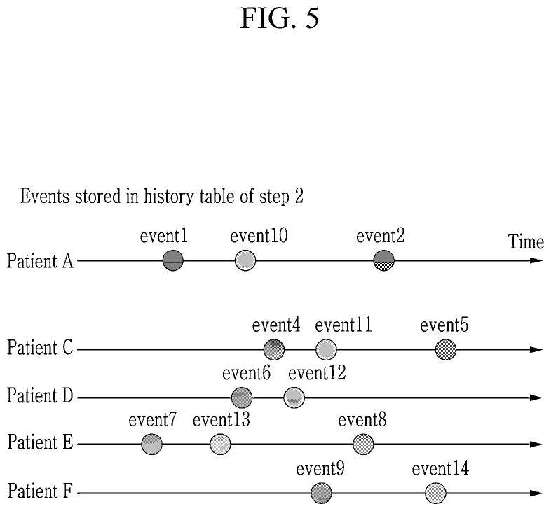

Referring to FIG. 5, the cohort extracting apparatus 100 receives input of the criteria (drug) of step 2, and generates history table 2 including events that satisfy the criteria of step 2 from history table 1.

The cohort extracting apparatus 100 identify, among the patients included in history table 1 of step 1, patients in a current step having an event corresponding to the criteria (drug) of step 2 by referring to the clinical data warehouse (CDW). The cohort extracting apparatus 100 then updates the bit string of all events of the current step patients recorded in history table 1 of step 1 (for example, updates the bit string from “0000000001” to “0000000011”) and adds the events extracted in step 2 as new events to generate history table 2 of step 2. At this time, the cohort extracting apparatus 100 identifies, among the patients included in history table 1, a patient (previous step patient) who does not have any one event corresponding to the criteria (drug) of step 2, and excludes the events of the previous step patient without retrieving the events into the history table of step 2.

For example, it is assumed that among the patients included in history table 1, patient B has no event that corresponds to the criteria (drug) in step 2. It is assumed that event10 to event14 are newly extracted in step 2. The cohort extracting apparatus 100 may then generate history table 2, as shown in Table 3. The number of patients recorded in history table 2 is 5 and the number of events is 13.

TABLE 3

| event | person_ID | visit_ID | event_type | criteria_type |
|---|---|---|---|---|
| 1 | A | 1 | 0000000011 | |
| New 10 | A | 2 | 0000000010 | drug |
| 2 | A | 3 | 0000000011 | |
| 4 | C | 7 | 0000000011 | |
| New 11 | C | 8 | 0000000010 | drug |
| 5 | C | 9 | 0000000011 | |
| 6 | D | 11 | 0000000011 | |
| New 12 | D | 12 | 0000000010 | drug |
| 7 | E | 13 | 0000000011 | |
| New 13 | E | 14 | 0000000010 | drug |
| 8 | E | 15 | 0000000011 | |
| 9 | F | 17 | 0000000011 | |
| New 14 | F | 18 | 0000000010 | drug |

Referring to Table 3, patient B does not have an event that corresponds to the criteria (drug) in step 2, so event3 that is patient B's hypertension diagnosis event is not recorded in history table 2.

The event type “0000000001” of event1, event2, event4, to event9 included in history table 1 are the events of the current step patient whose event corresponds to the criteria (drug) of step 2, so the bit string is updated to “0000000011” in which the second digit assigned to step 2 is 1.

The newly extracted event10 to event14 in step 2 are added to history table 2, and the event type of the newly extracted events is written as “0000000010” in which the second digit from the end assigned in step 2 is 1. Also, since event10 to event14 were first extracted from the criteria in step 2, drug is written in the detailed criteria type (criteria_type).

When the cohort extracting apparatus 100 receives a request for the number of events extracted in step 2, the cohort extracting apparatus 100 may calculate the number of rows in history table 2 that have the event type (event_type) of “0000000010” and output the number of events as 5.

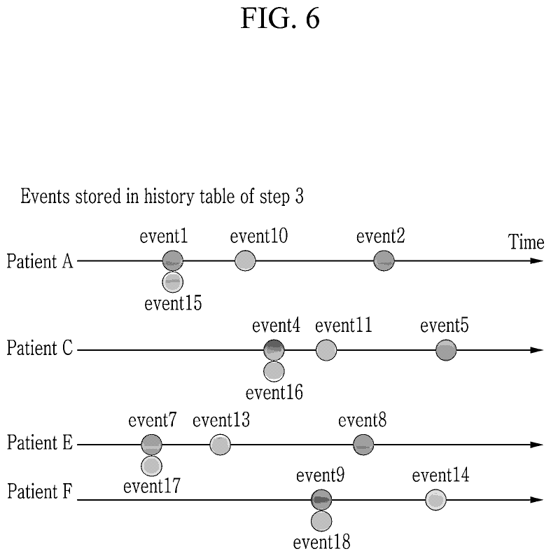

Referring to FIG. 6, the cohort extracting apparatus 100 receives input of the criteria of step 3, and generates history table 3 including events that satisfy the criteria of step 3 from history table 2.

The cohort extracting apparatus 100 identifies, among the patients included in history table 2 of step 2, a current-step patient having an event corresponding to the criteria of step 3 by referring to the clinical data warehouse (CDW). The cohort extracting apparatus 100 then updates the bit string of the event for the current step patient recorded in history table 2 of step 2 (for example, updates from “0000000011” to “0000000111”). Then, the cohort extracting apparatus 100 may add the new events extracted in step 3 to history table 3 of step 3.

When there is a previous step patient that does not correspond to the criteria of step 3 among the patients included in history table 2, the cohort extracting apparatus 100 deletes events for the patient.

On the other hand, when the criteria is age/gender, the criteria for calculating age/gender may be the earliest event, the latest event, and each event of the patient.

For example, it is assumed that among the patients included in history table 2, patient D does not correspond to the criteria (age) for step 3, and the remaining patients are current-step patients who satisfy the criteria for step 3. The cohort extracting apparatus 100 may then generate history table 3 that does not include event6 and event12 of the previous step patient, patient D, as shown in Table 4. The cohort extracting apparatus 100 updates the bit string of the events for the current step patient recorded in history table 2 of step 2. The bit string is updated with a 1 in the third digit assigned to step 3.

In addition, the cohort extracting apparatus 100 adds the new events extracted in step 3 to history table 3 of step 3, and when the age calculation criteria is the earliest event of the patient, new event15, new event16, new event 17, and new event18, which have the same event identifiers as the earliest events event1, event4, event7, and event9 of patient A, patient C, patient E, and patient F, respectively, may be added to history table 3, as shown in Table 4. Then, the cohort extracting apparatus 100 writes age in the detailed criteria type (criteria_type) of new event15, new event16, new event17, and new event18.

TABLE 4

| | | | event_type | |
|---|---|---|---|---|
| event | person_ID | visit_ID | (bit string) | criteria_type |
| 1 | A | 1 | 0000000111 | |
| New 15 | A | 1 | 0000000100 | age |
| 10 | A | 2 | 0000000110 | drug |
| 2 | A | 3 | 0000000111 | |
| 4 | C | 7 | 0000000111 | |
| New 16 | C | 7 | 0000000100 | age |
| 11 | C | 8 | 0000000110 | drug |
| 5 | C | 9 | 0000000111 | |
| 7 | E | 13 | 0000000111 | |
| New 17 | E | 13 | 0000000100 | age |
| 13 | E | 14 | 0000000110 | drug |
| 8 | E | 15 | 0000000111 | |
| 9 | F | 17 | 0000000111 | |
| New 18 | F | 17 | 0000000100 | age |
| 14 | F | 18 | 0000000110 | drug |

On the other hand, event15, event16, event17, and event18 extracted by the criteria of age/gender have the same event identifiers (visit identifier, event start date, and event end date) as event1, event4, event7, and event9, so when the number of events is calculated, the events extracted by the criteria of age/gender may be excluded from the number of events. Thus, the number of patients recorded in history table 3 is 4, and the number of events may be calculated as 11. The cohort extracting apparatus 100 may identify events whose detailed criteria type is age/gender (criteria_type=‘age’, criteria_type=‘gender’) in each history table and exclude the identified events from the total number of events.

In this way, the cohort extracting apparatus 100 generates a history table including the events for each patient at each step, and updates the bit string indicating whether the criteria is satisfied for each event in the history table. Thus, the cohort extracting apparatus 100 may quickly calculate the number of patients and the number of events at each step by utilizing a plurality of history tables, without having to write a SQL query each time when searching for the number of patients satisfying the criteria. In specific, it is possible to quickly check the step at which the event was extracted and the step at which the event satisfies the criteria through the bit string displayed for the event type.

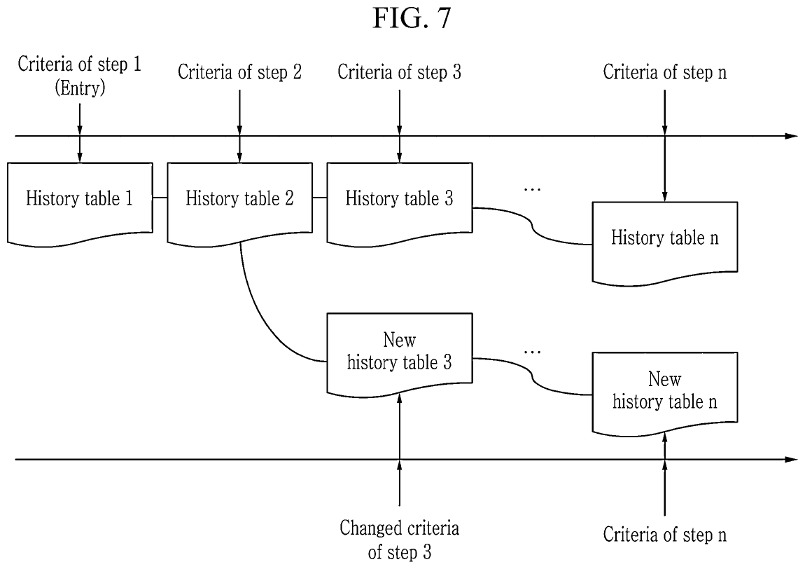

FIG. 7 is a diagram illustrating a cohort re-extracting method using a history table.

Referring to FIG. 7, the cohort extracting apparatus 100 first generates a history table 1 for the cohort entry criteria, and then separately generates history table 2, . . . , and history table n by using the input criteria in steps.

Subsequently, when the researcher changes the criteria of step k (for example, step 3), the cohort extracting apparatus 100 may generate new history table 3 corresponding to the changed criteria of step 3 by using history table 2 from the just previous step, step 2. The cohort extracting apparatus 100 may sequentially regenerate the history tables of the steps after step 3 by using re-generated new history table 3.

In this way, when a researcher changes criterion, the researcher still uses the history table as it was before the change and only needs to extract events for the changed criteria, so that a cohort extraction speed may be improved.

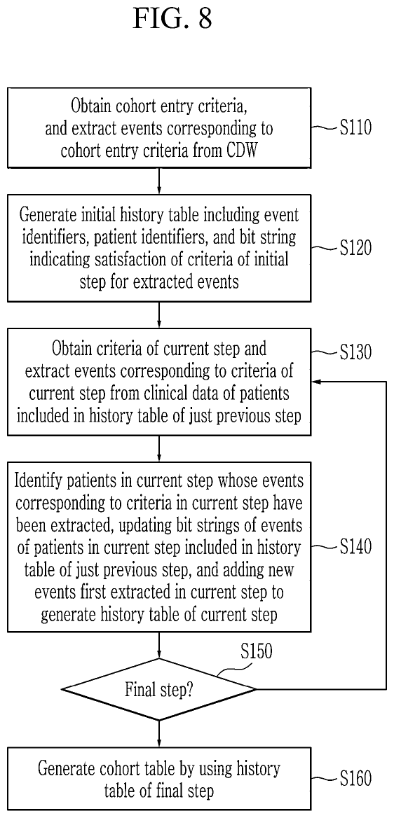

FIG. 8 is a flowchart illustrating the cohort extracting method.

Referring to FIG. 8, the cohort extracting apparatus 100 obtains cohort entry criteria in an initial step and extracts events corresponding to cohort entry criteria from the clinical data warehouse (CDW) (S110).

The cohort extracting apparatus 100 generates an initial history table including event identifiers (visit identifier, event start date, event end date), patient identifiers, and a bit string indicating satisfaction of the criteria of the initial step for the extracted events (S120).

Thereafter, the cohort extracting apparatus 100 obtains criteria of the current step and extracts events corresponding to the criteria of the current step from clinical data of the patients included in the history table of the just previous step (S130).

The cohort extracting apparatus 100 identifies patients in the current step whose events correspond to the criteria in the current step have been extracted from patients included in the history table of the just previous step, updates the bit strings of the events of the patients in the current step included in the history table of the just previous step, and adds new events first extracted in the current step to generate a history table of the current step (S140). The cohort extracting apparatus 100 identifies, among the patients included in the history table of the just previous step, previous step patients who do not have events corresponding to criteria in the current step, and does not store events of the previous step patients stored in the history table of the just previous step in the history table of the current step.

The cohort extracting apparatus 100 determines whether the current step is the final step (S150). When the current step is not the final step, the cohort extracting apparatus 100 waits in the state of being capable of receiving input of the criteria of the next extraction step. When the cohort extracting apparatus 100 receives a request to terminate or generate a cohort table, the cohort extracting apparatus 100 may determine that the current step is the final step.

When the current step is the final step, the cohort extracting apparatus 100 generates a cohort table by using the history table of the final step (S160). In this way, the cohort extracting apparatus 100 sequentially generates the history tables in steps, and then generates a cohort table by using the history table of the final step.

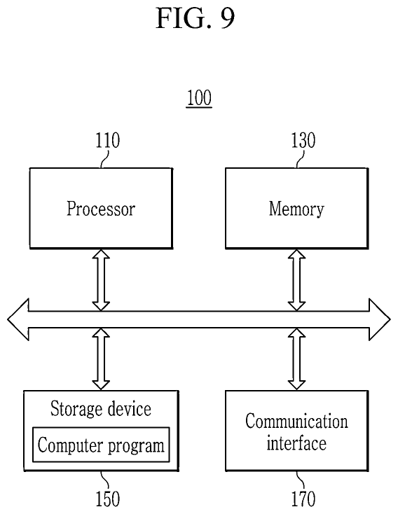

FIG. 9 is a hardware diagram of a computing apparatus according to an exemplary embodiment.

Referring to FIG. 9, the cohort extracting apparatus 100 may be implemented as a computing apparatus operated by at least one processor.

The cohort extracting apparatus 100 may include one or more processors 110, a memory 130 for loading a computer program performed by the processor 110, a storage apparatus 150 for storing the computer program and various data, and a communication interface 170. In addition, the cohort extracting apparatus 100 may further include various other components.

The processor 110 is a apparatus that controls the operation of the cohort extracting apparatus 100 and may be various forms of processor that processes instructions contained in a computer program, and may include, for example, at least one of a Central Processing Unit (CPU), a Micro Processor Unit (MPU), a Micro Controller Unit (MCU), a Graphic Processing Unit (GPU), or any other form of processor well known in the art of the present disclosure.

The memory 130 stores various data, instructions, and/or information. The memory 130 may load a corresponding computer program from the storage apparatus 150 such that the instructions described to execute the operations of the present disclosure are processed by the processor 110. The memory 130 may be, for example, Read Only Memory (ROM) and Random Access memory (RAM).

The storage apparatus 150 may nontemporarily store computer programs and various data. The storage apparatus 150 may include a non-volatile memory, such as a Read Only Memory (ROM), an Erasable Programmable ROM (EPROM), an Electrically Erasable Programmable ROM (EEPROM), a flash memory, or the like, a hard disk, a removable disk, or any other form of computer-readable recording medium well known in the art to which the present disclosure belongs.

The communication interface 170 may be a wired/wireless communication module that supports wired/wireless communication. The communication interface 170 may access the CDW 20.

A computer program may include instructions executed by the processor 110, and may be stored on a non-transitory computer readable storage medium, and the instructions cause the processor 110 to execute the operation of the present disclosure. The computer program may be downloaded through a network or sold as a product.

The computer program may include instructions for receiving input of a cohort entry criteria, extracting events satisfying the cohort entry criteria from a clinical data warehouse (CDW), and generating an initial history table including event information, a patient identifier, and a bit string indicating whether the criteria is satisfied up to a current step for the extracted events. Further, the computer program may include instructions for receiving input of criteria of the current step, identifying, among patients included in a history table of a just previous step, a current step patient having an event corresponding to the criteria of the current step, updating a bit string of the event of the current step patient included in the history table of the just previous step, and adding the event extracted from the current step as a new event to generate a history table of the current step. The program may include instructions for determining whether the current step is the final step, and, when the current step is the final step, generating a cohort table by using a history table of the final step. The computer program may include instructions for waiting in a state of capable of receiving input of criteria of a next extraction step when the current step is not the final step.

The exemplary embodiments of the present disclosure described above are not only implemented through the apparatus and method, but may also be implemented through programs that realize functions corresponding to the configurations of the exemplary embodiment of the present disclosure, or through recording media on which the programs are recorded.

Although an exemplary embodiment of the present disclosure has been described in detail, the scope of the present disclosure is not limited by the exemplary embodiment. Various changes and modifications using the basic concept of the present disclosure defined in the accompanying claims by those skilled in the art shall be construed to belong to the scope of the present disclosure.

1. A method of operating a cohort extracting apparatus, the method comprising:
obtaining cohort entry criteria and extracting events corresponding to the cohort entry criteria from a clinical data warehouse;
generating an initial history table including an event identifier, a patient identifier, and a bit string indicating satisfaction of criteria of an initial step for each extracted event;
obtaining criteria of a current step, identifying, among patients included in a history table of a just previous step, current step patients having an event corresponding to the criteria of the current step, and updating a bit string for each event of the current step patients included in the history table of the just previous step, and generating a history table of the current step by adding new events extracted in the current step; and
sequentially generating a history table for each step, and then generating a cohort table by using a history table of a final step.

2. The method of claim 1, wherein each history table generated in each step
includes events that satisfy criteria of a corresponding step, and is written with an event identifier, a patient identifier, and a bit string indicating whether the criteria have been satisfied up to the corresponding step for each event, and
the bit string is assigned digits indicating 1 or 0 whether the condition of each step is satisfied.

3. The method of claim 1, wherein the generating of the history table of the current step includes:
checking the events of the current step patients in the history table of the just previous step;
updating bit strings of the checked events with a value representing the satisfaction of the criteria of the current step; and
recording the updated bit string in the history table of the current step.

4. The method of claim 1, wherein the generating of the history table of the current step includes
when a new event is extracted from the current step, recording an identifier of the new event, a patient identifier, and a bit string indicating the satisfaction of the criteria of the current step in the history table of the current step, and
wherein the bit string of the new event is written with a value of 1 for digit specified in the current step, and a value of 0 for digits specified in other steps.

5. The method of claim 1, wherein the generating of the history table of the current step includes
among the patients included in the history table of the just previous step, identifying a previous step patient who does not have an event corresponding to the criteria of the current step, and not recording events of the previous step patient in the history table of the current step.

6. The method of claim 1, further comprising:
when a request for the number of events or the number of patients extracted from a specific step is received, calculating the number of events or the number of patients by using a history table of the specific step.

7. The method of claim 1, further comprising:
obtaining changed criteria of a specific step;
retrieving a just previous step history table generated in a just previous step of the specific step; and
identifying, among patients included in the just previous step history table, specific step patients having events corresponding to the changed criteria of the specific step, updating a bit string for each event of the specific step patients included in the just previous step history table, and regenerating a history table of the specific step by adding new events extracted in the specific step.

8. The method of claim 7, further comprising:
sequentially regenerating a history table of a step after the specific step by using the regenerated history table of the specific step.

9. A method of operating a cohort extracting apparatus, the method comprising:
obtaining criteria;
based on clinical data of patients included in a first history table generated in a just previous step, identifying a current step patient satisfying the criteria among the patients included in the first history table;
recording event identifiers, a patient identifier, and an updated bit string of all events of the current step patient included in the first history table, in a second history table;
when a new event corresponding to the criteria is extracted, recording an event identifier of the new event, a patient identifier, and a bit string representing the event extracted in the current step in a second history table; and
storing the second history table as a history table of the current step.

10. The method of claim 9, wherein, for all events of the current step patient included in the first history table, a bit string in which a value of a digit specified to the current step in the bit string recorded in the first history table is updated to 1 is recorded in the second history table.

11. The method of claim 9, wherein, for the new event, a bit string in which a value of a digit specified to the current step is 1 and a value of a digit specified in other steps is 0 is recorded in the second history table.

12. The method of claim 9, wherein, among the events included in the first history table, events of a previous step patient having no event corresponding to the criteria are not recorded in the second history table.

13. A computer program stored in a computer-readable storage medium and comprising instructions for causing at least one processor to execute:
obtaining cohort entry criteria and extracting events corresponding to the cohort entry criteria from a clinical data warehouse;
generating an initial history table including an event identifier, a patient identifier, and a bit string indicating satisfaction of criteria of an initial step for each extracted event;
obtaining criteria of a current step, identifying, among patients included in a history table of a just previous step, current step patients having an event corresponding to the criteria of the current step, and updating a bit string for each event of the current step patients included in the history table of the just previous step, and generating a history table of the current step by adding new events extracted in the current step; and
sequentially generating a history table for each step, and then generating a cohort table by using a history table of a final step.

14. The computer program of claim 13, wherein:
each history table generated in each step
includes events that satisfy criteria of a corresponding step, and is written with an event identifier, a patient identifier, and a bit string indicating whether the criteria have been satisfied up to the corresponding step for each event, and
the bit string is assigned digits indicating 1 or 0 whether the condition of each step is satisfied.

15. The computer program of claim 13, wherein the generating of the history table of the current step includes:
checking the events of the current step patients in the history table of the just previous step, updating bit strings of the checked events with a value representing the satisfaction of the criteria of the current step, and recording the updated bit string in the history table of the current step; and
when a new event is extracted from the current step, recording an identifier of the new event, a patient identifier, and a bit string indicating the satisfaction of the criteria of the current step in the history table of the current step.
